(19) United States
(12) Patent Application Publication
(10) Pub. No.: US 2012/0122201 A1
(43) Pub. Date: May 17, 2012

(54) Gas Flow Rate And Liquid Level Monitoring Apparatus

(75) Inventors: Stephen James Butler, Berkshire (GB); Stephen Mark Joseph Wilkins, London (GB); David Wingate, Surrey (GB)

(73) Assignee: Planer PLC, Middlesex (GB)

(21) Appl. No.: 13/384,767

(22) PCT Filed: Jul. 12, 2010
(86) PCT No.: PCT/GB2010/051139

§ 371 Date: Jan. 19, 2012

(30) Foreign Application Priority Data
Jul. 20, 2009 (GB) 0912552.7

(51) Int. Cl. C12M 1/00; G01F 13/00

(52) U.S. Cl. 435/303.1; 73/861.41

(57) ABSTRACT

An incubator (10) comprising an incubator housing (14), at least one sealable incubation chamber (16) in the housing (14), means for controlling temperature, humidity and gas composition within the incubation chamber (16), a liquid container (26), and a window (24) for observing the gas flow rate and liquid level of said a liquid container (26), said container having at least one light transmissible side wall (38), at least one gas inlet conduit (28) having a gas outlet at or adjacent to the said at least one light transmissible side wall (38), a gas outlet conduit (32) having a gas inlet which is spaced above the gas outlet of the said at least one gas inlet conduit (28), and a light emitting device (34) for directing a beam of light to be incident with or adjacent to an interior surface (46) of the said at least one light transmissible side wall (38) so as to in use illuminate at least one gas bubble (44) discharged from the gas outlet.

The present invention relates to gas flow rate and liquid level monitoring apparatus.

Laboratory incubators are widely used, and the internal atmosphere must be carefully regulated for temperature, humidity and gas composition. The gas composition for such incubators is typically controlled by introducing external gas from outside the incubator. Humidity is controlled by pumping this gas through water held in a container.

However, constant checks involving manually opening the cover above the water container have to be undertaken to make sure that the external gas is still being adequately introduced and that a sufficient amount of water in the container remains. Repeated opening of this cover causes temperature fluctuations, which may be detrimental to the contents.

The present invention seeks to provide a solution to these problems, and also a solution which is applicable to any equipment requiring humidity and gas composition control by the use of gas flowing in a pipe or conduit.

According to a first aspect of the present invention, there is provided gas flow rate and liquid level monitoring apparatus comprising a liquid container having at least one light transmissible side wall, at least one gas inlet conduit having a gas outlet at or adjacent to the said at least one light transmissible side wall, at least one gas outlet conduit having a gas inlet which is spaced above the gas outlet of the said at least one gas inlet conduit, and a light emitting device for directing a beam of light to be incident with or adjacent to an interior surface of the said at least one light transmissible side wall so as to in use illuminate at least one gas bubble discharged from the gas outlet.

Preferable and/or optional features of the first aspect of the invention are set forth in claims 2 to 20, inclusive.

According to a second aspect of the present invention, there is provided an incubator comprising an incubator housing, at least one sealable incubation chamber in the housing, means for controlling temperature, humidity and gas composition within the incubation chamber, and gas flow rate and liquid level monitoring apparatus in accordance with the first aspect of the invention.

Preferable and/or optional features of the second aspect of the invention are set forth in claims 23 to 26, inclusive.

According to a third aspect of the present invention, there is provided an incubator comprising an incubator housing, at least one sealable incubation chamber in the housing, means for controlling temperature, humidity and gas composition within the incubation chamber, a liquid container, at least one gas inlet conduit for discharging gas into the liquid container, at least one gas outlet conduit for the flow of gas out of the liquid container, and a conduit guide element for guiding portions of the gas inlet conduit(s) and the gas outlet conduit(s) outside of the liquid container.

Preferable and/or optional features of the third aspect of the invention are set forth in claims 29 to 34, inclusive.

The invention will now be more particularly described, by way of example only, with reference to the accompanying drawings, in which:

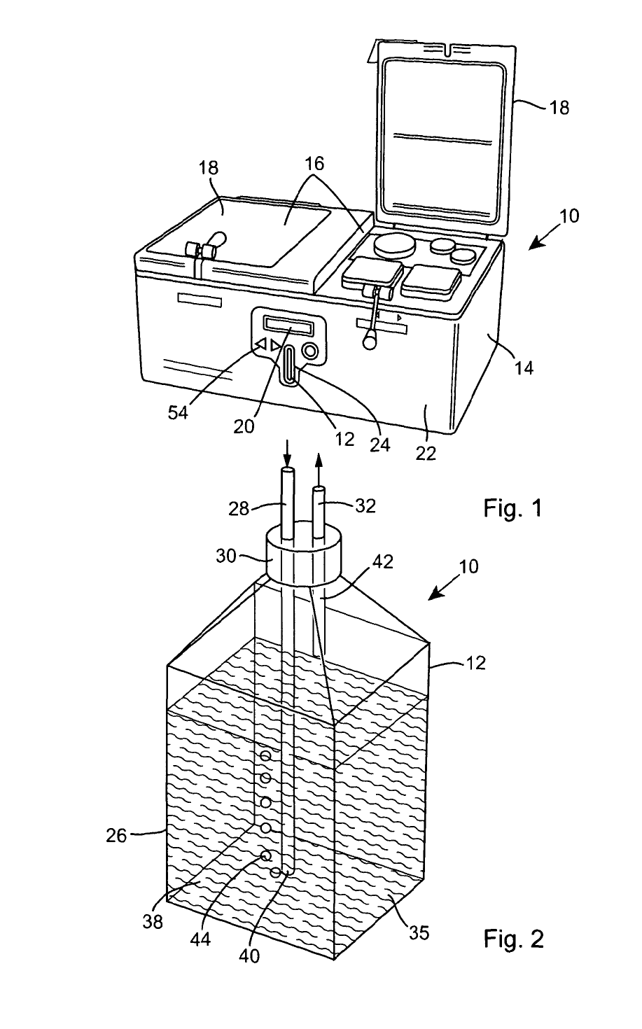

FIG. 1 is a perspective view of a laboratory incubator having a first embodiment of gas flow rate and liquid level monitoring apparatus, in accordance with the first aspect of the invention;

FIG. 2 shows a diagrammatic representation of an internal liquid container which is viewable through a front wall of the incubator, shown in FIG. 1;

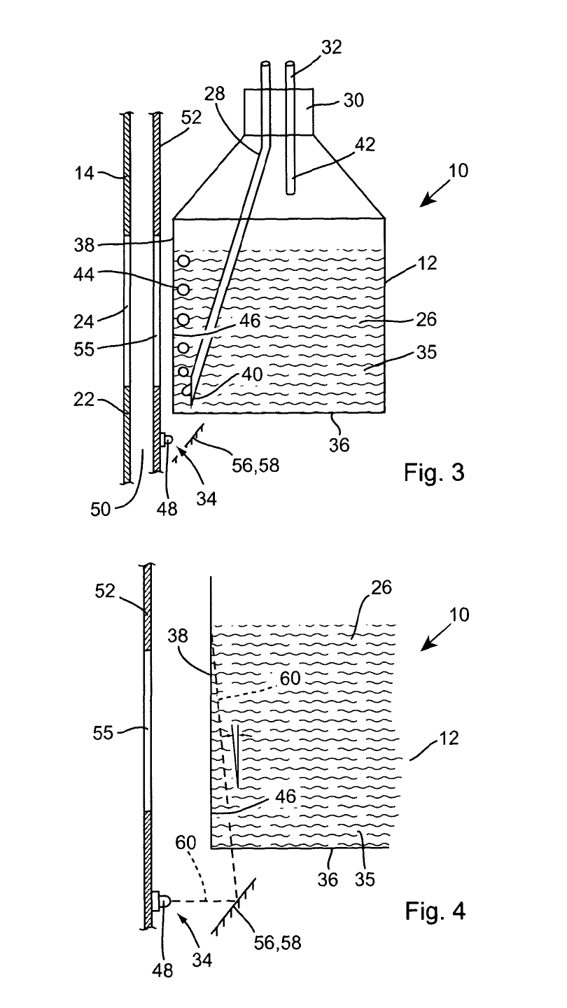

FIG. 3 shows a side elevational view of the internal container and the front wall of the incubator in cross-section;

FIG. 4 diagrammatically shows a preferred positioning of a reflective element and an angle of reflection;

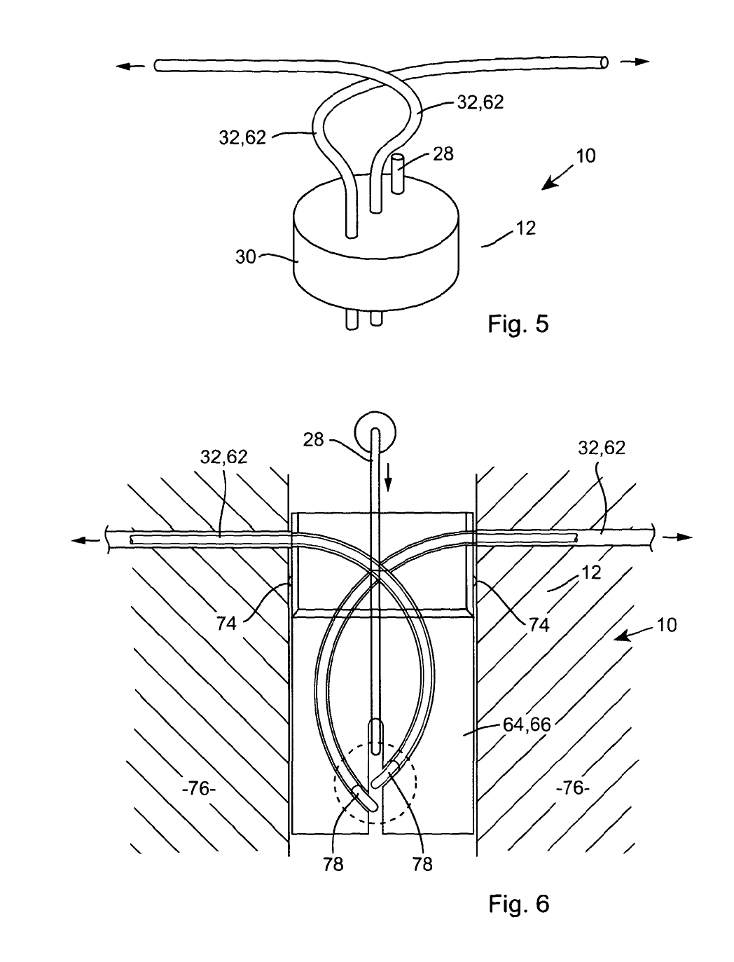

FIG. 5 shows part of a second embodiment of gas flow rate and liquid level monitoring apparatus, in accordance with the first aspect of the invention and showing arcuate conduits;

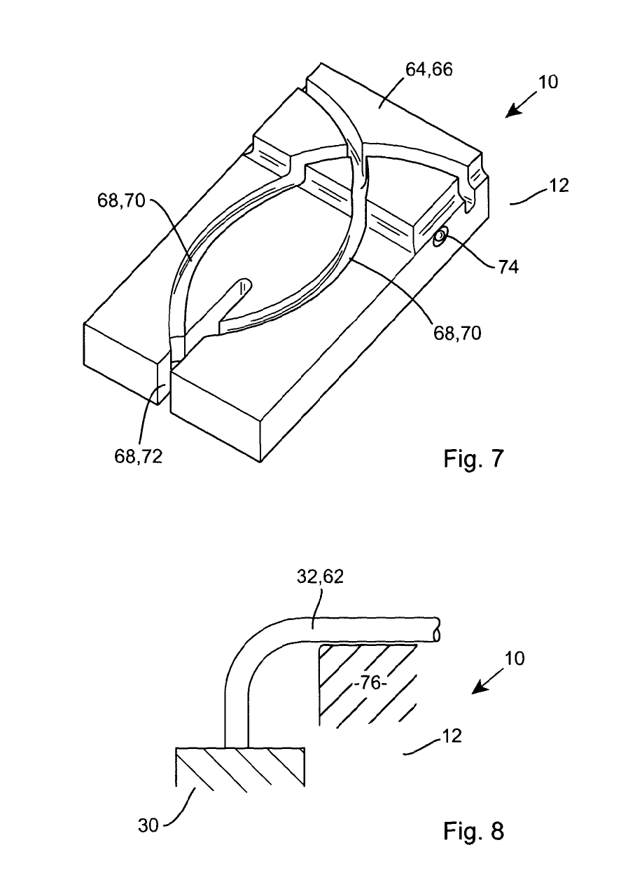

FIG. 6 shows a guide element for the conduits of the apparatus shown in FIG. 5;

FIG. 7 shows a perspective view of the guide element; and

FIG. 8 shows part of the guide element when in use and guiding a conduit; and

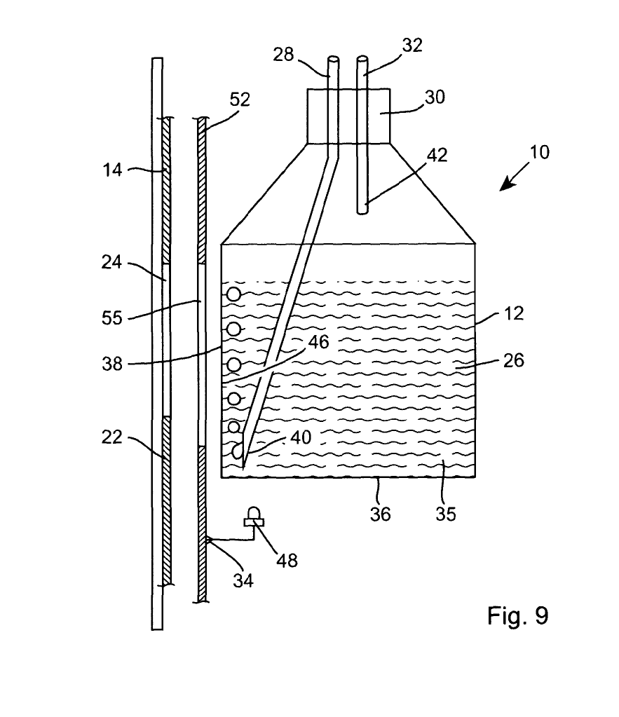

FIG. 9 is a diagrammatic view of a third embodiment of gas flow rate and liquid level monitoring apparatus, in accordance with the first aspect of the invention and showing another position of a light emitting element.

Referring firstly to FIGS. 1 to 4 of the drawings, there is shown a laboratory incubator 10 having a first embodiment of gas flow rate and liquid level monitoring apparatus 12. The incubator 10 comprises a rigid incubator housing 14 having two separate and independently controllable incubator chambers 16 each closable by a separate access cover 18. A graphical display 20 is provided on a front surface, wall or panel 22 of the incubator housing 14, and below this is provided a window 24 forming part of the gas flow rate and liquid level monitoring apparatus 12.

FIGS. 2 to 4 show simplified details of the gas flow rate and liquid level monitoring apparatus 12, which includes a rigid, preferably plastics or glass, liquid container 26 held internally within the incubator housing 14 and which is provided at or adjacent to the front surface window 24, a gas inlet conduit 28 which extends a first distance into the liquid container 26 from a top 30 thereof, a gas outlet conduit 32 which extends a second distance into the liquid container 26 which is less than the first distance, and a light emitting device 34 which in this embodiment is positioned below a bottom surface 36 of the liquid container 26.

At least one side surface or wall 38 of the liquid container 26 is light transmissible, preferably being transparent, so that the contents of the liquid container 26 can be viewed from outside the incubator housing 14. The liquid container 26 itself is preferably removable to permit periodic cleaning and sterilisation.

Beneficially, the liquid container 26 is closed with the only ingress and egress being via the gas inlet conduit 28 and the gas outlet conduit 32. By provision of a filter (not shown) in the gas inlet conduit 28, the contents of liquid container 26 may be maintained sterile compared to the liquid container 26 being open. The liquid container 26 and the gas inlet and outlet conduits 28 and 32 are also fully enclosed within the housing 14 which is beneficial in helping to maintain the container 26 and conduits 28, 32 at desired and constant temperatures.

This is an improved arrangement because the conduits 28, 32 can be kept warm and so condensation is less likely. Also, the liquid container 26 is fully enclosed within the incubator housing 14 and therefore has its temperature better maintained.

Since Petri dishes or the like are typically incubated within the incubator 10 for several days, evaporation therefrom becomes a significant issue causing the concentration of chemicals in the solution or medium to increase. Consequently, preventing or limiting this evaporation is critical. Furthermore, an enclosed liquid container 26 with the gas inlet conduit 28 and the gas outlet conduit 32 therein enables a more uniform and temperature controllable environment within the liquid container 26, thus again reducing the possibility of condensation within the inlet and outlet conduits 28, 32.

The inlet and outlet conduits 28, 32 may be flexible plastic pipes, and by engaging these with the liquid container 26, accidental kinking or bending of the conduits 28, 32 and thus a reduced or halted flow of gas can occur.

The liquid container 26 is typically part filled with a liquid 35, such as sterilised water. The level of the liquid 35 is such that an inner end 40 of the gas inlet conduit 28 is below the liquid level and an inner end 42 of the gas outlet conduit 32 is above the liquid level.

The inner end 40 of the gas inlet conduit 28 terminates at or adjacent to the light transmissible wall 38 of the liquid 35 container 26, and in this embodiment this is achieved by cranking a longitudinal extent of a part of the gas inlet conduit 28 which extends within the liquid 35 container 26. The longitudinal extent of the part of the gas outlet conduit 32 which is within the liquid 35 container 26 is rectilinear, but it may be non-rectilinear.

Although cranked, the longitudinal extent of the part of the gas inlet conduit 28 which extends within the liquid 35 container 26 may be curved.

By positioning the inner end 40 of the gas inlet conduit 28 at or adjacent to the light transmissible wall 38, in use a flow of gas bubbles 44 discharged therefrom travels to the liquid surface substantially along the interior surface 46 of the light transmissible wall 38 of the liquid container 26, which are thus viewable from outside the incubator housing 14 via the window 24.

Generally, in a small incubator 10, the internal gas composition is maintained by supplying a small steady flow of premixed gas from an external source. This external source typically contains carbon dioxide, oxygen and nitrogen. The humidity of the inflowing gas is increased by bubbling it through the liquid 35, typically water, held in the liquid container 26 and mounted internally within the incubator housing 14. Gas is introduced to the liquid container 26 via the gas inlet conduit 28 at or adjacent to the bottom of the liquid container 26. On discharge, it flows upwards as a stream of bubbles 44 through the liquid 35 and to the surface. The gas which accumulates above the surface of the liquid 35 exits the liquid container 26 via the shorter gas outlet conduit 32.

However, it can be difficult to distinguish the stream of bubbles 44 from certain angles and distances away from the front panel or surface 22 of the incubator housing 14. Furthermore, distinguishing a change in flow rate of the bubbles 44 can also be difficult. Consequently, the aforementioned light emitting device 34 is utilised.

In this embodiment, the light emitting device 34 includes at least one light emitting element 48 which is provided in an interior 50 of the incubator housing 14. The light emitting element 48 may conveniently be an LED, since this utilises only a small amount of power thus maintaining battery backup life of the incubator 10, as well as emitting very little heat thus minimising an impact on the temperature regulation of the container 26.

The light emitting element 48 is mounted on an interior-facing surface of a PCB 52 which is positioned within the incubator housing 14 adjacent to the front wall 22. This positioning of the PCB 52 is additionally advantageous, since it allows mounting of a user control interface 54 which is accessible by a user from the front wall 22 of the incubator 10.

The light emitting element 48 is below the liquid container 26 and oriented so as to direct a beam of, preferably blue or green, light at right angles or substantially right angles to the light transmissible side wall of the liquid container 26.

The light emitting device 34 also includes a light reflective element 56 having a reflective surface 58 provided at an angle below the bottom surface 36 of the liquid container 26. By providing a light transmissible bottom surface 36, a light beam 60 can be discharged from the light emitting element 48 to enter the liquid container 26 via the light reflective surface 58 and the light transmissible bottom surface 36. Since the light reflective surface 58 is positioned substantially below the light transmissible side wall 38, the reflected light illuminates the bubble stream discharged from the gas inlet conduit 28.

The PCB 52 includes a corresponding aperture 55 which is aligned with the incubator window 24 so as to allow viewing of the liquid container 26. The light emitting element 48 and reflective element 56 are low enough so as not to be visible or not easily visible by an observer looking through the incubator window 24.

As shown in FIG. 4, it is preferable that the reflected light beam 60 is at an angle α to the light transmissible side wall 38 so that the light beam 60 is incident with the interior surface 46 of the light transmissible side wall 38 at or adjacent to the liquid surface. This results in additional illumination of the liquid surface meniscus, allowing a user or operator to easily monitor a surface level via the incubator window 24. In the event that a meniscus is not formed, this arrangement is still beneficial since a bubble 44 breaking the liquid surface tends to scatter light to a user's eye, allowing the liquid level to be readily ascertained.

No or substantially no further illumination of the liquid container 26 is provided so as to limit any possibility of bacterial growth in container 26. To this end, the window 24 and PCB aperture 55 are relatively narrow to limit the ingress of ambient light.

The incubator window 24 and/or the PCB aperture 55 may be open or closed. If closed, a light transmissible material is used, and this may be transparent or translucent. A filter may also be utilised to further limit the ingress of ambient light. In this latter case, preferably the filter is blue or green, since these colours inhibit algae growth. In this instance the filter colour and the colour of the light emitted by light emitting device 38 would be chosen to be the same in order for the reflected light to be transmitted through window 24 to the user.

Keeping the container 26 in the dark as much as possible is beneficial from the point of view of suppressing any growth of algae or similar notwithstanding the bottle and contents should be sterile in the first place. Any window, opening and/or aperture to the container 26 to allow viewing of the contents and hence the stream of bubbles must conversely let light into the container. Internal illumination as described above allows the window, opening and/or aperture to be only just sufficiently large enough to see the bubbles clearly and to minimise inward light transmission. This enables unwanted colours of light to be excluded.

Regarding the colour of the light being used to illuminate the bubbles, it would be possible to have a green or blue covered window, opening and/or aperture for admitting ambient light, but the light would be severely attenuated by passing through the covered window twice, after first entering the hosing 12 and then leaving. The use of internal coloured illumination is thus the preferred option.

Furthermore, as embryos are adversely sensitive to light, it is common for IVF labs which are typically where such incubators are placed to have quite low ambient light levels. The illumination afforded by this ambient light would therefore be typically insufficient for viewing the gas bubbles, and even more so behind a coloured filter.

Referring to FIGS. 5 to 8, there is shown part of a second embodiment of gas flow rate and liquid level monitoring apparatus 12, which comprises a modified top 30 of the liquid container 26. Two said gas outlet conduits 32 are provided, one for each incubator chamber 16, and the positioning of the inner ends 42 of the gas outlet conduits 32 is the same as described in the first embodiment so that a gas flow can be monitored. A single gas inlet conduit 28 is provided as described above.

The gas inlet and outlet conduits 28, 32 are typically formed from thin flexible tubular material, such as silicon, as mentioned above. The outer portions 62 which extend away from the liquid container 26 are thus prone to kinking and blocking by condensation.

In this embodiment, therefore, the gas flow rate and liquid level monitoring apparatus 12 includes a conduit guide element 64 having a solid guide body 66 with conduit grooves or channels 68 formed, for example by machining, therein. The channels 68 are dimensioned so that the gas inlet and outlet conduits 28, 32 are press-fittably insertable. The outlet channels 70 have a gently curving longitudinal extent for guiding the gas outlet conduits 32 and preventing kinking, and the inlet channel 72 is straight or rectilinear.

Spring plungers 74 are included which allows the conduit guide element 64 to rotate upwards to permit the end user to fit a new liquid container 26 as well as making it possible for the user to completely remove the conduit guide element 64 from the incubator 10 for maintenance and cleaning.

Abutments 76 are also included which extend above and to the sides of the liquid container 26 and which support the conduit guide element 64 via the spring plungers 74. Preferably, electrical heating elements are included as part of the apparatus 12 to heat the abutments 76. The conduit guide element 64 is thus heated by conduction and the conduits 28, 32 are warmed, thereby reducing a risk of condensation and conduit blockage.

The conduit guide element 64 includes two relieved portions 78 at lower ends of the inlet channels 68. This allows the gas outlet conduits 32 to follow a natural path from the conduit guide element 64 again without resulting in kinking.

Ideally, the temperature of container 26 and conduits 28, 32 would all be the same as incubator chambers 16 so the incoming gas would be at the same temperature as the incubator chambers 16 and would not disturb their temperature. The gas leaving container 26 via the gas outlet conduit 32 is always at 100% humidity having been bubbled through the water or liquid in the container 26, and this leaving gas is also at the temperature of container 26.

However, if conduits 28, 32 are even slightly cooler than container 26, condensation will occur which could block the gas flow therein. Therefore container 26 is preferably kept cooler than incubator chambers 16, so the gas entering and leaving container 26 via gas inlet and outlet conduits 28, 32 is if anything going to be warmed therein, thereby preventing or limiting the risk of condensation.

Likewise the gas entering incubator chamber 16 will be warmed again and once more to prevent or limit condensation. Therefore, ideally the gas leaving container 26 at 100% humidity is warmed in conduits 28, 32 and warmed again on entering incubator chamber 16.

If these conditions are met there will be no condensation, but the humidity of the gas entering the incubation chamber 16 will be less than 100%, due to the warming.

Ideally, the container 26 and conduits 28, 32 are kept as uniformly close in temperature as possible to the incubator chamber 16 but a small temperature increment is preserved from container 26 to conduits 28, 32 and again to incubator chamber 16. The apparatus 10 with the temperature controlled guide elements 64 provides gas at as high an humidity as it is possible to achieve without condensation problems. Without temperature controlled guide element 64 this is virtually impossible to achieve because otherwise the conduits 28, 32 are suspended between the container 26 and the incubator chamber(s) 16 and therefore cannot be kept at a higher temperature than container 26.

FIG. 9 shows a third embodiment of gas flow rate and liquid level monitoring apparatus 12 which is similar to the first embodiment. Consequently, similar references refer to similar parts, and further detailed description is omitted.

In this embodiment, the light emitting element 48 is moved from the interior surface 46 of the PCB 52 so as to be oriented to shine directly upwards through the light transmissible bottom surface 36 of the liquid container 26. As such, the light reflective surface of the first embodiment is dispensed with.

In the above embodiments, the outlet opening of the gas inlet conduit is preferably canted relative to the longitudinal extent of the inner portion of the gas inlet conduit.

Although the conduit guide element and abutments are described in conjunction with the viewing window of the incubator and the gas flow rate and liquid level monitoring apparatus, the conduit guide element and abutments with heating device can be utilised in an incubator independently of the gas flow rate and liquid level monitoring apparatus. In this case, the liquid container would still preferably be provided along with the gas inlet and outlet conduits. However, the viewing window and thus thus light emitting device may be dispensed with.

The conduit guide element may feasibly be utilised without the heater, for example, simply making use of insulation. Although conductive heating is preferred, other forms of heating, such as convection and/or infra-red could be considered.

The guide element is also preferably rigid. However, a guide element which allows some flex whilst still providing reasonably firm support and direction can be considered.

The guide element, although preferably pivotable, may be fixed. It may also be permanently retained in the incubator housing, rather than being removable.

The means for controlling temperature, humidity and gas composition within the incubation chamber mentioned herein is standard equipment and thus readily available to the skilled address. As such, the details thereof have not been described in any detail or shown in the drawings.

It is thus possible to provide gas flow rate and liquid level monitoring apparatus which allows external visual monitoring of a gas flow rate whilst being incorporated as part of gas composition and humidity control apparatus within the incubator. Implementation of the gas flow rate and liquid level monitoring apparatus is thus simple and cost-effective. The gas monitoring apparatus also prevents or limits condensation and kinking of the gas inlet and outlet conduits. It is also possible to provide an incubator with the gas flow rate and liquid level monitoring apparatus. It is further possible to provide an incubator with a conduit guide element which guides gas flow conduits within the incubator to prevent or limit kinking, whilst also preferably providing heating of the conduits.

The embodiments described above are provided by way of examples only, and further modifications will be apparent to persons skilled in the field without departing from the scope of the invention as defined by the appended claims.

1-35. (canceled)

36. An incubator comprising an incubator housing, at least one sealable incubation chamber in the housing, at least one controller which controls at least one of temperature, humidity and gas composition within the incubation chamber, a liquid container, at least one gas inlet conduit which discharges gas into the liquid container, at least one gas outlet conduit which enables the flow of gas out of the liquid container, and a conduit guide element which guides portions of the gas inlet conduit(s) and the gas outlet conduit(s) outside of the liquid container.

37. An incubator as claimed in claim 36, wherein the conduit guide element includes a rigid arcuate guide channel which guides the or each gas outlet conduit.

38. An incubator as claimed in claim 36, wherein the conduit guide element includes a rigid rectilinear guide channel which guides the or each gas inlet conduit.

39. An incubator as claimed in claim 37, wherein two said arcuate guide channels are provided to accommodate two gas outlet conduits.

40. An incubator as claimed in claim 37, wherein the outlet conduit is press-fittable in its respective guide channel.

41. An incubator as claimed in claim 36, wherein the conduit guide element is pivotable.

42. An incubator as claimed in claim 36, wherein the conduit guide element is removable.

43. An incubator as claimed in claim 36, further comprising a heater which heats the conduit guide element.

44. Gas flow rate and liquid level monitoring apparatus comprising a liquid container having at least one light transmissible side wall, at least one gas inlet conduit having a gas outlet at or adjacent to the said at least one light transmissible side wall, at least one gas outlet conduit having a gas inlet which is spaced above the gas outlet of the said at least one gas inlet conduit, and a tight emitting device which directs a beam of light to be incident with or adjacent to an interior surface of the said at least one light transmissible side wall so as to in use illuminate at least one gas bubble discharged from the gas outlet.

45. An incubator comprising an incubator housing, at least one sealable incubation chamber in the housing, at least one controller which controls at least one of temperature, humidity and gas composition within the incubation chamber, and gas flow rate and liquid level monitoring apparatus as claimed in claim 44.
